(19) United States
(12) Patent Application Publication
(10) Pub. No.: US 2026/0096829 A1
(43) Pub. Date: Apr. 9, 2026

(54) SURGICAL INSTRUMENT WITH METHODS FOR OVER-CLAMPING PREVENTION

(71) Applicant: Cilag GmbH International, Zug (CH)

(72) Inventors: Yi Zhou Cheng, Stanford, CA (US); Sree Shankar Satheesh Babu, San Jose, CA (US); Aedan M. Mangan, Cincinnati, OH (US); Dimitrios Chatzigeorgiou, Redwood City, CA (US)

(21) Appl. No.: 18/910,069

(22) Filed: Oct. 9, 2024

(51) Int. Cl. A61B 17/29; A61B 34/30; A61B 90/00

(52) U.S. Cl. CPC A61B 17/29; A61B 34/30; A61B 90/06; A61B 2034/305; A61B 2090/064

(57) ABSTRACT

A surgical system that prevents over-clamping includes a surgical instrument with a shaft assembly, an end effector, and a closure assembly. The end effector includes first and second jaws movably secured relative to each other so that the first and second jaws are configured to selectively move from an open to closed configuration. The closure assembly is operatively connected to the end effector and comprises a drive member operatively connected to the end effector and configured to selectively move in an open or closed direction to respectively direct one of the first and second jaws from the open to closed configuration or from the closed to open configuration, a sensor portion configured to measure a sensed value of a characteristic of the drive member, and a controller configured to receive the sensed value evaluate the received sensed value, and adjust the movement of the drive member based on the evaluation.

### BACKGROUND

A medical operator, during a robotic assisted surgery, may leverage a variety of surgical instruments, to include a tissue grasper and a tissue sealer. The surgical instruments perform functions for the operator such as grasping a blood vessel, cutting tissue, coagulating tissue, or dissecting tissue. When the function requires a grasping, clamping, pinching, or other action that holds or presses parts together firmly, closure force is an important consideration. Closure force may be known as clamp force during vessel sealing, grip force during tissue manipulation or suturing, and closure force during stapling. If the closure force is too great, or unstable across different instrument apertures, bodily harm or damage to the surgical instrument may occur—an occurrence known as over-clamping.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; U.S. Pat. No. 11,058,493, entitled “Robotic System Configured for Navigation Path Tracing,” issued Jul. 13, 2021, the disclosure of which is incorporated by reference herein, in its entirety; and U.S. patent application Ser. No. 18/170,688, entitled “Surgical Instrument with Clamp Closure Compensation and Related Methods,” filed Feb. 17, 2023, the disclosure of which is incorporated by reference herein, in its entirety.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a control console and a plurality of robotic arms;

FIG. 2 depicts a perspective view of an example of a robotic arm, an example of a tool driver, and a first example of a surgical instrument, each configured for use with the table-based robotic system of FIG. 1;

FIG. 3A depicts an enlarged schematic perspective view of the tool driver and the surgical instrument of FIG. 2;

FIG. 3B depicts a schematic perspective view of the tool driver similar to FIG. 3A, but with the surgical instrument removed to expose rotary drives;

FIG. 4 depicts a perspective view of a second example of a surgical instrument configured to use with the table-based robotic system of FIG. 1 as well as the robotic arm and the tool driver of FIG. 2;

FIG. 5 depicts an enlarged perspective view of a distal end portion of the surgical instrument of FIG. 4 with an end effector in an open configuration and an articulation section in a straight configuration;

FIG. 6 depicts an enlarged perspective view of the distal end portion of the surgical instrument of FIG. 4 with the end effector in the open configuration and rolled about a longitudinal axis;

FIG. 7 depicts an enlarged perspective view of the distal end portion of the surgical instrument of FIG. 4 with the end effector in the open configuration and the articulation section articulated to adjust a pitch of the end effector through a pitch plane;

FIG. 8 depicts an enlarged perspective view of the distal end portion of the surgical instrument of FIG. 4 with the end effector in the open configuration and the articulation section articulated to adjust a yaw of the end effector through a yaw plane;

FIG. 9 depicts an enlarged perspective view of the distal end portion of the surgical instrument of FIG. 4 with the end effector in a closed configuration and the yaw of the end effector adjusted through the yaw plane;

FIG. 10A depicts an enlarged perspective view of the distal end portion of the surgical instrument of FIG. 4 with a portion of the end effector in broken lines for greater clarity of a knife member in a proximal position;

FIG. 10B depicts an enlarged perspective view of the distal end portion of the surgical instrument similar to FIG. 10A, but with the knife member in a distal position;

FIG. 11 depicts an enlarged rear schematic view of a proximal end portion of the surgical instrument of FIG. 4 with a plurality of cables configured to direct manipulation of the end effector;

FIG. 12 depicts an enlarged perspective view of the distal end portion of the surgical instrument of FIG. 4 with portions removed for greater clarity of a plurality of cables configured to direct manipulation of the end effector;

FIG. 13A depicts a side schematic view of a clamp closure system with the end effector of FIG. 4. directed to a fully open configuration and tissue in the end effector;

FIG. 13B depicts the side schematic view of the clamp closure system with the end effector similar to FIG. 13A, but being directed toward the closed configuration with tissue in the end effector;

FIG. 13C depicts the side schematic view of the clamp closure system with the end effector similar to FIG. 13B, but in the closed configuration with tissue in the end effector;

FIG. 14 depicts a flowchart of an example of a method of over-clamping prevention for an open-loop clamp closure system;

FIG. 15 depicts a flowchart of an additional example of a method of over-clamping prevention for an open-loop clamp closure system;

FIG. 16 depicts a flowchart of an example of a method of over-clamping prevention for a closed-loop clamp closure system; and

FIG. 17 depicts a side schematic view of the end effector of the surgical instrument of FIG. 4, highlighting the location on the end effector associated with different terms that comprise a closure force model for a closed-loop clamp closure system.

### DETAILED DESCRIPTION

### I. Overview of Example Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing, for example, bronchoscopy, ureteroscopy, and gastroscopy.

In addition to performing a breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Example of Robotic System Table

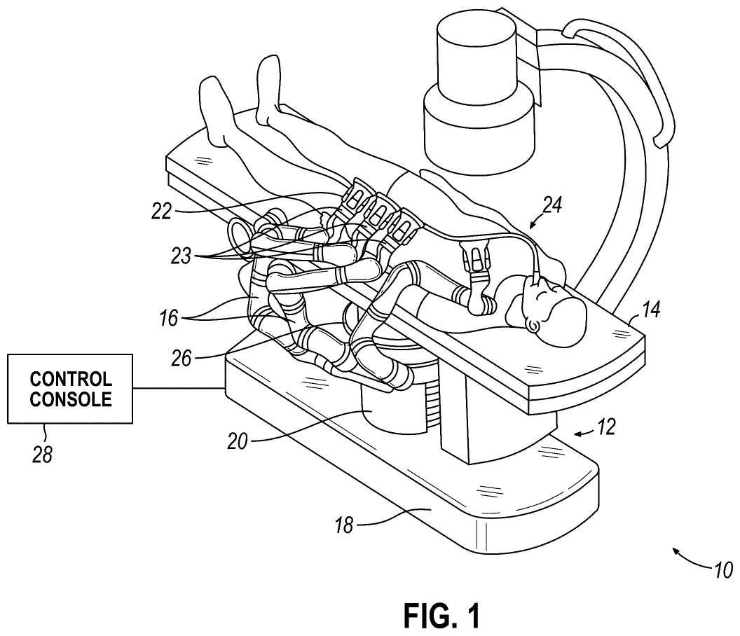

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) of the present example also includes a ring-shaped carriage (26), from which robotic arms (16) are based. Column (20) structurally supports platform (14) and provides a path for vertical translation of the carriage. In some versions, a table base may stow and store robotic arms (16) when not in use. A control console (28) is coupled with robotic surgical system (10). While four robotic arms are shown, more or fewer robotic arms are envisioned.

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable location. Tool drivers (22) are positioned at distal ends of robotic arms (16) in the present example. Tool drivers (22) are operable to manipulate one or more surgical instruments (24), as will be described in greater detail below.

### B. Example of Robotic Arm, Tool Drive, and Tool

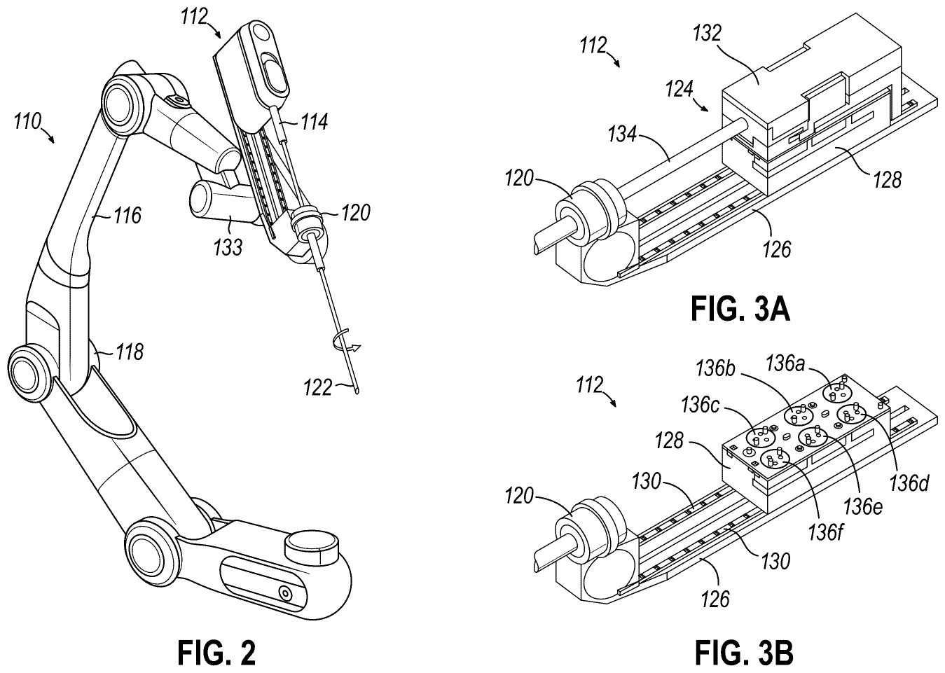

FIG. 2 shows an example of a robotic arm (110), a tool driver (112), and a surgical instrument (114), which may be incorporated into robotic surgical system (10) in place of a robotic arm (16), a tool driver (22), and a surgical instrument (24) that are shown in FIG. 1. Additional examples of robotic arms, tool drivers, and surgical instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) is attached to the distal end of robotic arm (110). Tool driver (112) includes a cannula (120) coupled to the end of tool driver (112), to receive and guide surgical instrument (114). Surgical instrument (114) may include an endoscope, a laparoscope, a stapler, graspers, an ultrasonic instrument, an RF electrosurgical instrument, or any other suitable kind of instrument. Surgical instrument (114) is inserted into the patient via cannula (120). The distal end of surgical instrument (114) includes an end effector (122). End effector (122) is configured to interact with the patient (e.g., providing visualization, stapling, grasping, ultrasonic cutting and/or sealing, electrosurgical cutting and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct a surgical instrument (114) from a desired point in space while allowing the clinician to move joints (118) into a clinically advantageous position away from the patient to create greater access, while avoiding collisions of robotic arms (110).

FIGS. 3A and 3B show tool driver (112) with and without a tool driver adapter (124), which may also be referred to as a tool base. As shown in FIGS. 3A and 3B, tool driver (112) may include a stage (126) and a carriage (128). Stage (126) includes longitudinal tracks (130). Carriage (128) is slidingly engaged with longitudinal tracks (130). Stage (126) of tool driver (112) may be configured to couple to a distal end (133) of robotic arm (110) such that articulation of robotic arm (110) positions and/or orients tool driver (112) in space. Surgical instrument (114) includes a tool driver adapter (124) at a proximal end and, as noted above, end effector (122) at a distal end. Tool driver adapter (124) includes a handle (132) and a shaft assembly (134) that extends distally from handle (132).

Carriage (128) is configured to couple with tool driver adapter (124). Carriage (128) may drive a set of articulated movements of end effector (122) and/or otherwise actuate end effector (122), such as through a cable system or wires manipulated and controlled by actuated drives. Carriage (128) may include different configurations of actuated drives, including but not limited to motorized rotary axis drives. The plurality of rotary axis drives may be arranged in any suitable manner. As shown in FIG. 3B, carriage (128) of the present example includes six rotary drives (136a-f) arranged in two rows, extending longitudinally along the base of carriage (128). Rotary drives (136a-c) are arranged in a first row that is longitudinally offset from a second row in which rotary drives (136d-f) are arranged. This staggered arrangement of rotary drives (136a-f) may reduce the width of carriage (128) and thereby provide a more compact form factor for tool driver (112). However, rotary drives (136a-f) may be provided in any other suitable arrangement. Moreover, any other suitable kind(s) of drive outputs may be provided by carriage (128), in addition to or in lieu of rotary drives (136a-f).

### II. Example of Surgical Instruments

### A. Overview

Robotic surgical system (10) includes a limited number of robotic arms (16, 110) onto which scopes and other surgical instruments may be coupled. Given the space constraints of robotic laparoscopic instrumentation, there are competing demands for use of these limited number of robotic arms (16, 110). For example, a surgeon may not want to dedicate a robotic arm to a surgical instrument that provides only suction, only irrigation, or only electrosurgical energy. As a result, it is beneficial for a surgical instrument coupled with one of robotic arms (16, 110) to perform multiple functions during the course of a surgical procedure. Additionally, having a surgical instrument that performs multiple functions reduces or eliminates surgical time associated with exchanging a first surgical instrument with a second surgical instrument providing a different function or capability.

### B. Example of a Surgical Instrument

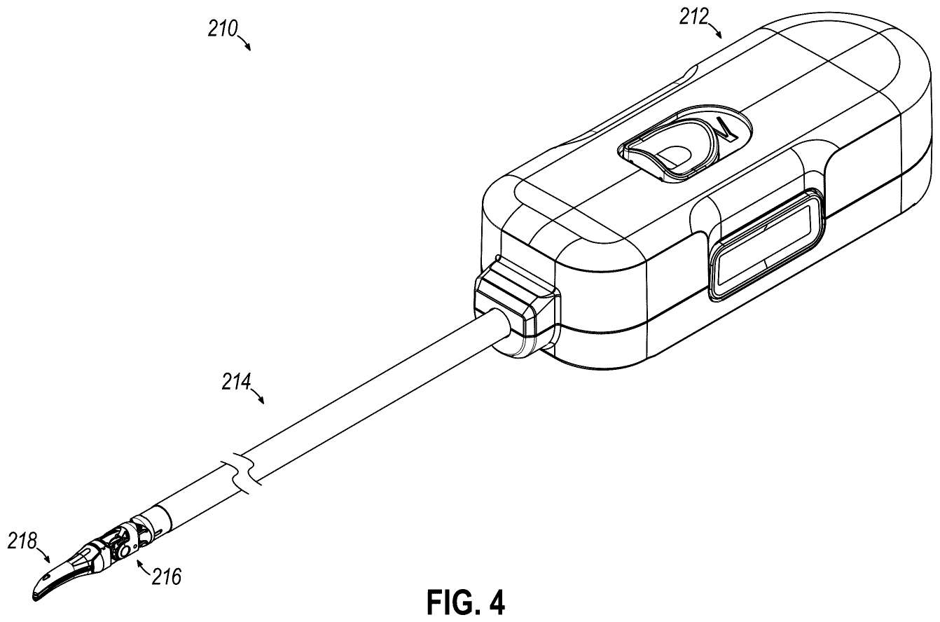

FIG. 4 shows a second example of a surgical instrument (210) configured for use with robotic surgical system (10) including robotic arms (16, 110) and tool drivers (22, 112) of FIGS. 1-2. Surgical instrument (210) may be used in place of surgical instrument (24, 114). As previously described, tool driver (112) includes stage (126) and carriage (128), with carriage (128) being configured to move relative to stage (126) to move surgical instrument (210) relative to patient (P). Surgical instrument (210) includes a body (shown as a tool drive adapter (212)), a shaft assembly (214), an articulation section (216) of shaft assembly (214), and an end effector (218).

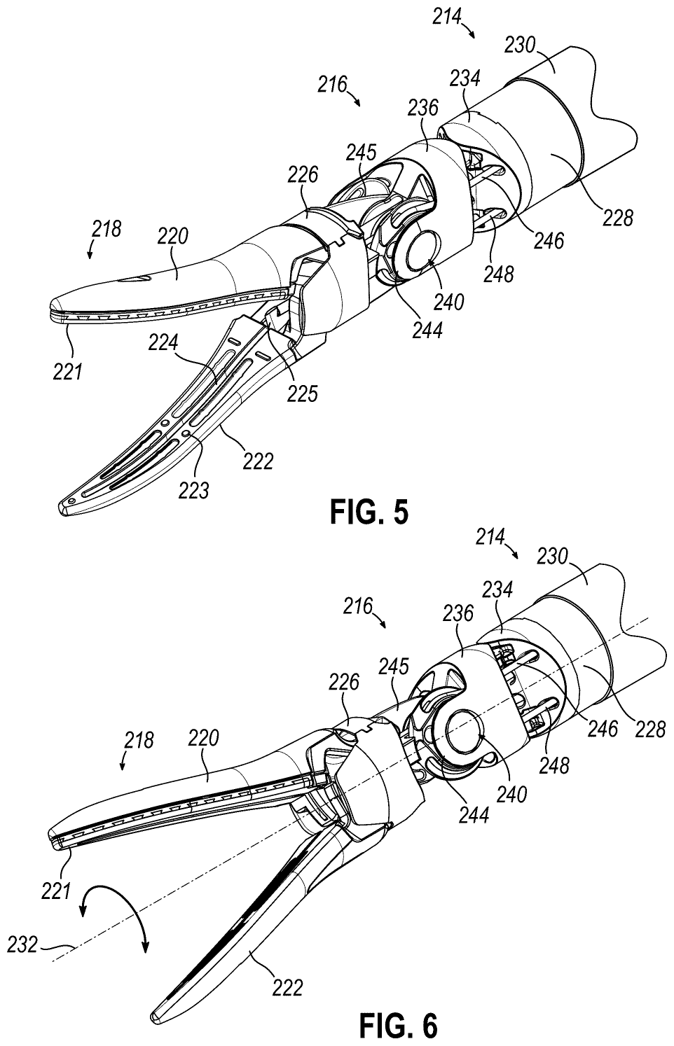

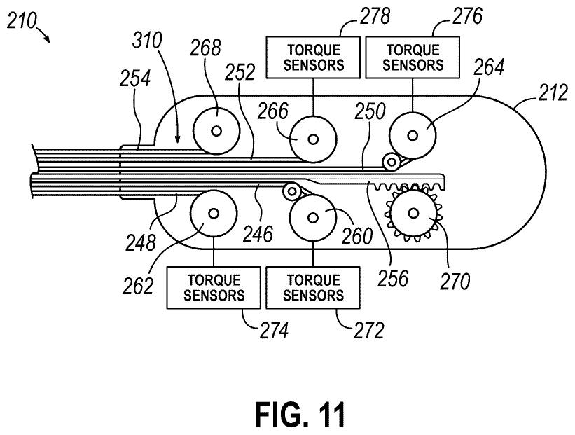

Turning to FIGS. 5-6, end effector (218) includes an upper jaw (220) having an upper electrode surface (221), a lower jaw (222) having a lower electrode surface (223), and a knife member (225) slidably disposed within a knife channel (224) cooperatively defined by upper jaw (220) and lower jaw (222). As will be described in greater detail below, end effector (218) of the current example is configured to grasp tissue with jaws (220, 222), seal tissue by applying bipolar RF energy to tissue via electrodes (221, 223), and sever tissue via knife member (225). In the current example, end effector (218) is suitably coupled to drive inputs (260, 262, 264, 266, 268, 270) (see FIG. 11) via cables and pulleys in order to suitably actuate components of end effector (218) in accordance with the description herein.

Upper jaw (220) and lower jaw (222) are pivotally coupled to each other such that jaws (220, 222) may actuate between an open configuration and a closed configuration in order to grasp tissue. In the current example, jaws (220, 222) are operatively attached to a clevis assembly (226) configured to translate to thereby pivot jaws (220, 222) between the open configuration and the closed configuration. While clevis assembly (226) is utilized in the current example, any other suitable structures may be utilized in order to drive jaws (220, 222) between the open and closed positions as would be apparent to one skilled in the art in view of the teachings herein.

Articulation section (216) extends between end effector (218) and an inner shaft (228) of shaft assembly (214). Inner shaft (228) is received within an outer shaft (230) of shaft assembly (214) and configured to selectively rotate about a longitudinal axis (232) within outer shaft (230). In turn, articulation section (216) and end effector (218) are rotatably fixed relative to inner shaft (228) such that articulation section (216) and end effector (218) similarly selectively rotate with inner shaft (228). Such rotation about the longitudinal axis (232) may also be referred to herein as “roll” or “rolling” to position articulation section (216) and end effector (218) as desired for improved angles to manipulate end effector (218) as discussed below in greater detail.

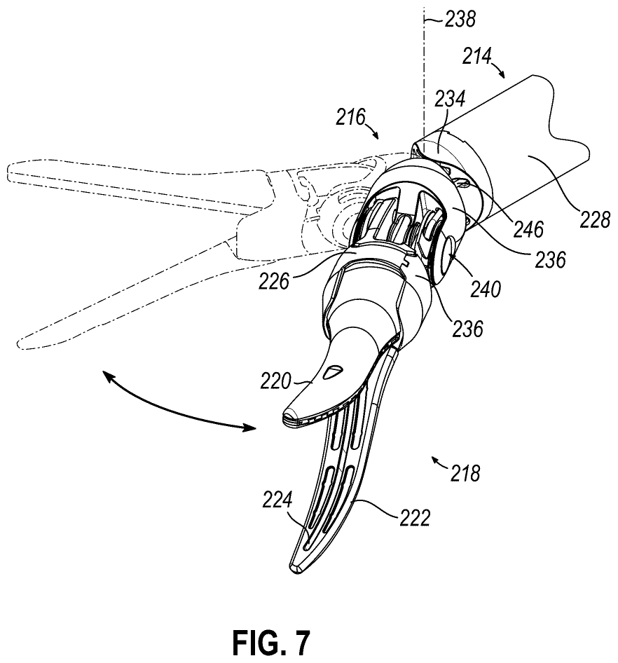

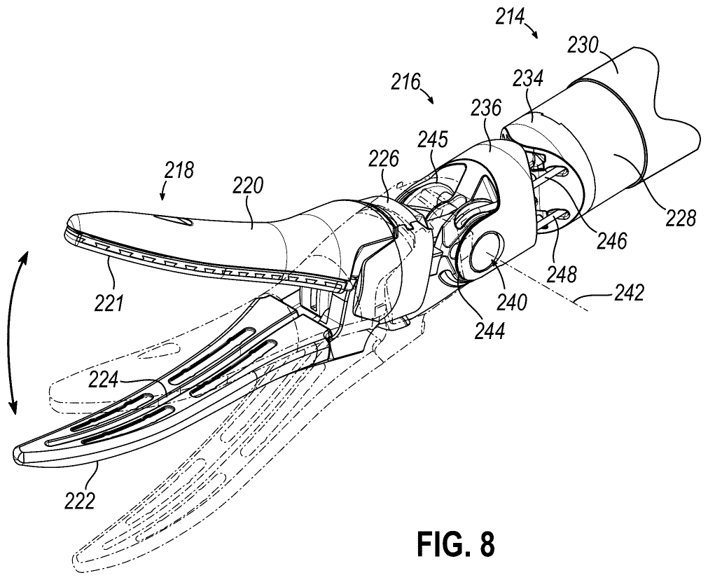

To this end, articulation section (216) is configured to articulate pitch relative to inner shaft (228) and further articulate yaw relative to inner shaft (228) to deflect end effector (218) respectively through a pitch plane as shown in FIG. 7 and a yaw plane as shown in FIG. 8. In the present example, articulation section (216) includes a proximal camming body (234) associated with a distal end of inner shaft (228) and a distal camming body (236) proximally projecting relative to end effector (218). Camming bodies (234, 236) are configured to engage each other as jaws (220, 222) deflect about a pitch axis (238), as shown in FIG. 7.

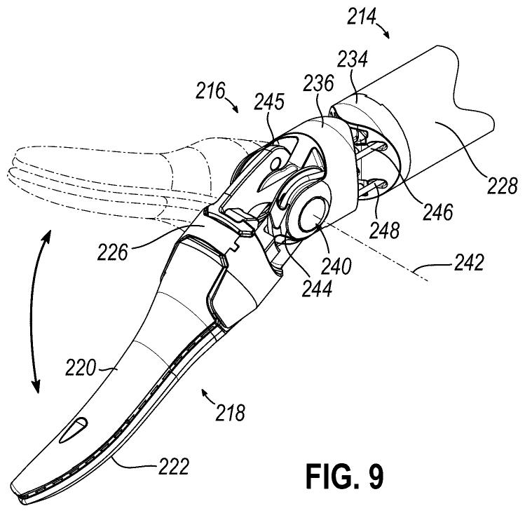

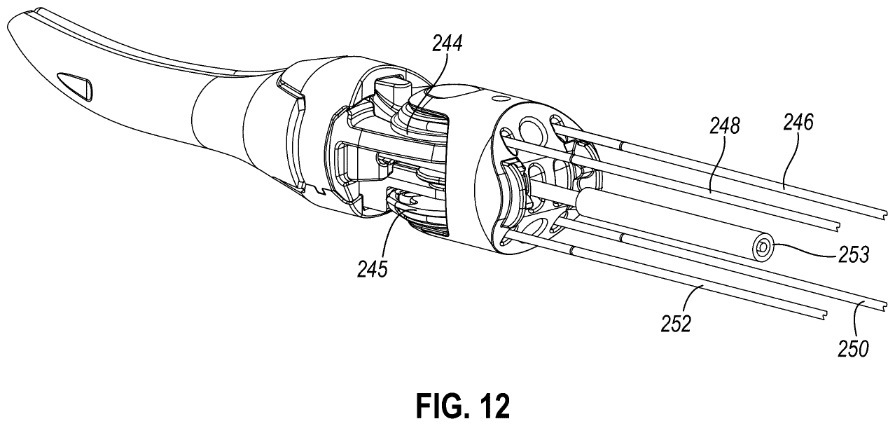

Articulation section (216) further includes a pivot coupling (240) such that jaws (220, 222) are pivotally coupled to a distal end portion of distal camming body (236). In this respect, pivot coupling (240) is configured to pivot jaws (220, 222) about a yaw axis (242), as shown in FIG. 9. Pivot coupling (240) more particularly includes a pair of pulleys (244, 245) and a plurality of drive cables (246, 248, 250, 252) shown in FIGS. 11 and 12, which are configured to be collectively driven to pivot jaws (220, 222) together through yaw plane for repositioning jaws (220, 222) as desired. Pulleys (244, 245) and cables (246, 248, 250, 252) are further configured to be collectively driven to pivot jaws (220, 222) both together through pitch plane for repositioning jaws (220, 222) as desired and relative to each other between the open and closed configurations. An example of pitch, yaw, and jaw motion in relation to the collective driving, by either pulling (i.e., drawing) or providing slack to (i.e., releasing), the plurality of drive cables (246, 248, 250, 252) is shown below in Table 1. The collective driving may vary in degree and amount. In other words, each of the plurality of drive cables (246, 248, 250, 252) may be driven by the same or different amounts and is not limited by the combinations in Table 1.

TABLE 1
Example of Motion Control for Jaws

| | Pitch | | Yaw | | Jaw | |
|---|---|---|---|---|---|---|
| | + | − | + | − | Open | Close |
| Drive Cable (248) | Pull | Slack | Pull | Slack | Pull | Slack |
| Drive Cable (246) | Pull | Slack | Slack | Pull | Slack | Pull |
| Drive Cable (252) | Slack | Pull | Pull | Slack | Slack | Pull |
| Drive Cable (250) | Slack | Pull | Slack | Pull | Pull | Slack |

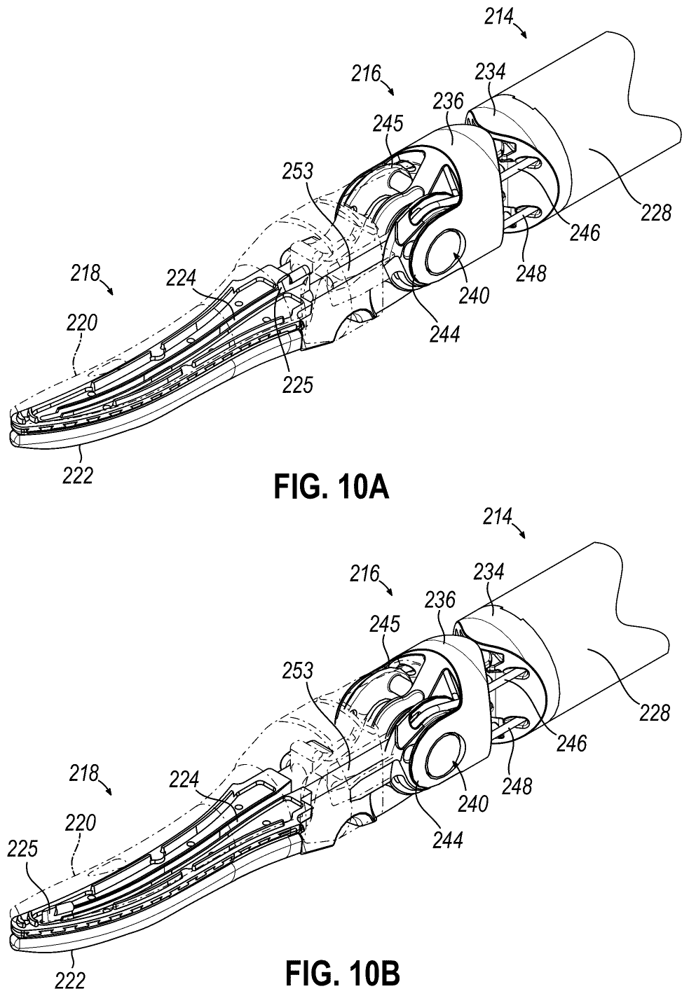

Referring back to FIGS. 10A-10B, while jaws (220, 222) are in the closed configuration, knife member (225) may be driven distally along a path defined by knife channel (224) from a proximal position (as illustrated in FIG. 10A) to a distal position (as illustrated in FIG. 10B) in order to sever tissue grasped by jaws (220, 222). Once knife member (225) reaches the distal position within knife channel (224) in order to suitably sever tissue, knife member (225) may then be retracted within knife channel (224) back into the proximal position. Notably, in the present example, knife member (225) distally extends from an elongate member (253), such as a nitinol tube, which is configured to translate through articulation section (216) for moving knife member (225) as discussed below in greater detail.

Electrode surfaces (221, 223) may be activated during any suitable time at which jaws (220, 222) interact with tissue in order to apply bipolar RF energy to tissue. For example, electrode surfaces (221, 223) may be activated after knife member (225) severs tissue in order to seal the recently severed tissue grasped between jaws (220, 222). As another illustrative example, electrode surfaces (221, 223) may be activated prior to knife member (225) severing tissue. As yet another illustrative example, electrode surface (221, 223) may be activated in order to cauterize tissue without cutting tissue.

In the current example, electrode surface (221) is an electrode body attached on an underside of jaw (220); while jaw (222) is formed from a suitable material in order to act as electrode surface (223). For example, jaw (222) may be formed of a metal material and be in connection with a ground wire; while electrode body forming electrode surface (221) is attached the underside of jaw (220) and in communication with a hot wire. Once suitably activated, RF energy may be transmitted between electrode surfaces (221, 223) in order to further transmit such RF energy through tissue.

Electrode surfaces (221, 223) may have any suitable configuration as would be apparent to one skilled in the art in view of the teachings herein. While in the current example, electrode surfaces (221, 223) are configured to deliver bipolar RF energy to tissue, it should be understood that end effector (218) may be configured to deliver any other suitable type of therapeutic energy to tissue as would be apparent to one skilled in the art in view of the teachings herein.

Articulation of articulation section (216) to deflect end effector (218) is directed by selectively moving associated cables (246, 248, 250, 252) as discussed briefly above, whereas roll of end effector (218) and movement of knife member (225) are directed by selectively moving associated members (254, 256). To this end, as shown in FIGS. 10B and 11, distal end portions of first upper cable (246) and first lower cable (248) attach to first pulley (244), whereas proximal end portions of first upper cable (246) and first lower cable (248) respectively attach to a first drive input (260) and a second drive input (262). Similarly, distal end portions of second upper cable (250) and second lower cable (252) attach to second pulley (245), whereas proximal end portions of second upper cable (250) and second lower cable (252) respectively attach to a third drive input (264) and a fourth drive input (266). Finally, in the present example, a fifth drive input (268) is operatively connected to inner shaft (228), articulation section (216), and end effector (218) in order to collectively direct roll of inner shaft (228), articulation section (216), and end effector (218) together, and a sixth drive input (270) is operatively connected to knife member (225) to direct movement of knife member (225) between proximal and distal positions. Such drive inputs may more particularly be referred to in one or more examples as capstans. Additional aspects of coordinating articulation of articulation section (216) as well as rolling end effector (218) and moving knife member (225) via cables (246, 248, 250, 252) are further discussed in U.S. Pat. No. 10,166,082, entitled “System and Method for Controlling a Robotic Wrist,” issued Jan. 1, 2019, the disclosure of which is incorporated by reference herein, in its entirety.

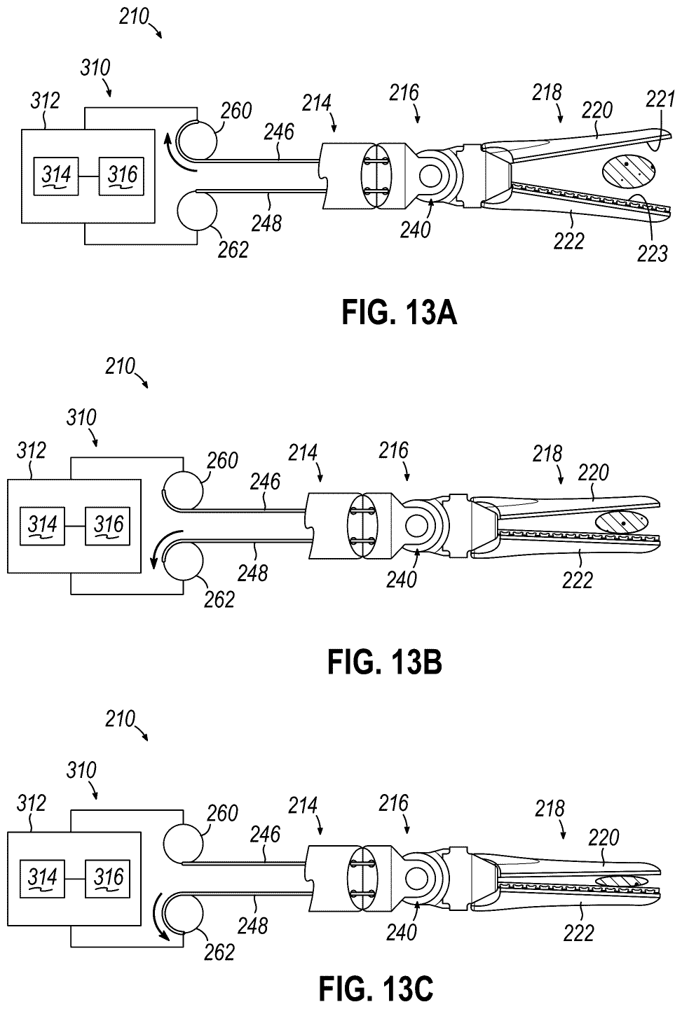

In some examples, certain drive inputs may be operatively connected to a sensor configured to sense a desired characteristic of the drive input as the drive inputs are moved in one of an open direction or a closed direction. Such desired characteristics may include, for example, a torque generated or applied by the drive input as well as the current draw of the drive input. As shown in FIG. 11, drive inputs (260, 262, 264, 266) are operatively connected to respective torque sensors (272, 274, 276, 278) configured to provide feedback to a CPU of a clamp closure system, such as CPU (314) of clamp closure system (310) seen in FIGS. 13A-13C, for use in over-clamping prevention methods described herein. In one example, torque sensors (272, 274, 276, 278) are positioned in carriage (128) (see FIG. 3B) and incorporated into rotary drives (126a-f) (see FIG. 3B) and configured to sense torque applied at drive inputs (260, 262, 264, 266). Of course, any such sensor configured to detect an aspect of torque, such as force, may be alternatively positioned for providing feedback of clamp force at jaws (220, 222) such that the invention is not intended to be unnecessarily limited to torque sensors (272, 274, 276, 278) shown in the present example.

### III. Clamp Closure Systems

FIGS. 13A-13C show an example of a clamp closure system (310) of surgical instrument (210) including a controller (312) having a central processing unit (CPU) (314) and a memory (316) configured to prevent an over-clamping clamp force, also known as closure force, from being applied with end effector (218) when tissue (e.g., blood vessel) is between jaws (220, 222) and jaws (220, 222) are being directed to a closed configuration. In this respect, like numbers indicate like features discussed above. Controller (312) may leverage open-loop or closed-loop control methods. The control methods and any associated configurations, thresholds, or settings may be stored in memory (316). Additionally, controller (312) may be incorporated into any portion of surgical system (10), such as surgical instrument (210) shown herein, and is not intended to be unnecessarily limited to incorporation into surgical instrument (210). More particularly, controller (312) is operatively connected to each of first and second drive inputs (260, 262) and configured to direct rotation of first and second drive inputs (260, 262) to in turn selectively direct draw and/or release of cables (246, 248) for moving jaws (220, 222). In this respect, FIGS. 13A-13C of the present example schematically show operation of first and second drive inputs (260, 262) and cables (246, 248) to direct jaws as described below, but it will be appreciated that additional inputs and cables, such as third and fourth drive inputs (264, 266) with cables (250, 252) may be similarly incorporated into clamp closure system (310). Also, articulation section (216) is generally shown in a straight configuration, but it will be appreciated that the following description of closing jaws (220, 222) similarly applies in one or more articulated configurations. The invention is thus not intended to be unnecessarily limited to the particular drive inputs (260, 262) and cables (246, 248) or a particular amount of articulation as discussed herein.

More particularly, FIG. 13A shows first drive input (260) drawing on upper cable (246) in tension to a fully open position with second drive input (262) and lower cable (248) remaining in a first position such that jaws (220, 222) are selectively directed to a fully open configuration via controller (312) to surround a vessel (30). To close jaws (220, 222), FIG. 13B shows first drive input (260) releasing upper cable (246) to a first position and second drive input (262) drawing on lower cable (248) to a second position such that jaws (220, 222) are selectively directed to a closed configuration via controller (312), with a clamp force starting to be exerted on the vessel (30). FIG. 13C shows the continuation of jaws (220, 222) closing on the vessel (30), and thereby increasing the clamp force being exerted on the vessel (30), as the jaws (220, 222) attempt to reach the closed configuration.

When the clamp closure system (310) applies an open-loop control method, the closed configuration is directed to the same location (e.g., a fully closed position where jaws (220, 222) are making contact with each other as shown in FIG. 9), which may be set during a calibration process, regardless of what is between jaws (220, 222). Accordingly, the larger the jaw aperture due to, for example, large tissue between jaws (220, 222), the greater the torque generated by the drive input that is drawing, second drive input (262) in FIGS. 13B-13C, as the drive input tries to reach the closed configuration due to the resistance of the larger tissue. In some cases, this increased torque can exceed the operational limits of certain components of the end effector (218), such as lower cable (248), leading to equipment failure and potential bodily harm. Equipment failure may include, for example, cables snapping or a clevis detaching. In yet other cases, the increased clamp force can be significant enough to harm the tissue between jaws (220, 222). Clamp force may also be affected by the presence of elongate member (253), which may be a nitinol tube, due to elongate member's (253) stiffness requiring additional draw from second drive input (262) when holding elongate member (253) at an articulated position.

### A. Open-Loop Clamp Closure System

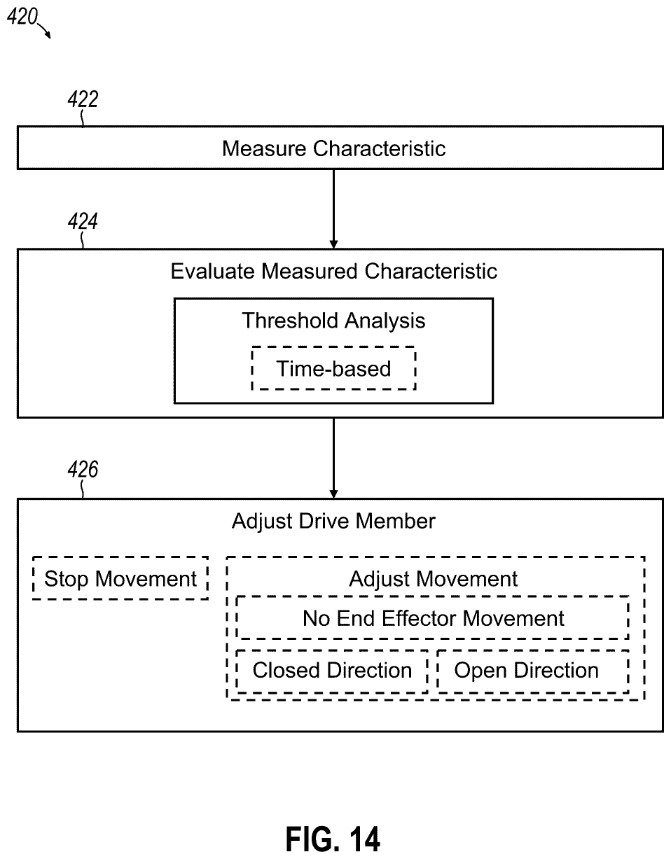

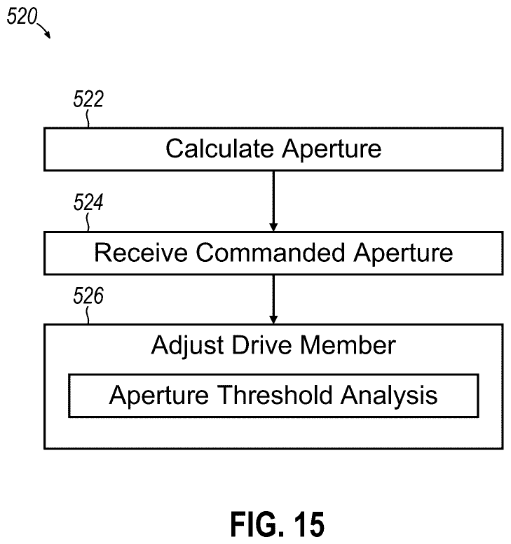

FIGS. 14-15 show examples of over-clamping prevention methods (420, 520) applicable to clamp closure system (310) suitable for an open-loop control method. In use, with respect to FIG. 14 and referring back to FIGS. 11, 13B, and 13C, a first example of a method of over-clamping prevention (420) includes measuring, at (422), a desired characteristic of the end effector (218). The characteristic may, in one example, be the torque generated by one or more drive inputs (260, 262, 264, 266) and sensed by one or more torque sensors (272, 274, 276, 278). In other words, one or more characteristic (e.g., torque) measurements may be used in over-clamping prevention method (420). Other characteristics, such as current draw, may be used and may incorporate additional sensors and/or different sensors.

Then, at (424), the measured characteristic is evaluated. Using torque as an example, a threshold analysis is performed on the one or more measured torques. In some examples, the threshold analysis may consist of comparing the one or more measured torques against a maximum torque threshold, which may be the torque when a predetermined cable tension is reached. The maximum torque threshold may be a single threshold or multiple thresholds, each of the multiple thresholds distinct for each drive input. In some examples, the maximum torque threshold may vary based on the position (e.g., the commanded pitch and yaw) of the end effector. When comparing the one or more measured torques against the maximum torque threshold, the threshold analysis may be considered satisfactory—that is, the threshold may be considered violated-if the one or more measured torques meets or exceeds the maximum torque threshold. Additionally, in some examples, the maximum torque threshold may be met or exceeded for a predetermined amount of time, or time-based, in order for the threshold analysis to be considered satisfactory.

If the threshold analysis is considered satisfactory, the method (420) proceeds to adjust (426) drive members, namely one or more of drive inputs (272, 274, 276, 278). In one example, the adjustment consists of stopping the directed movement (e.g., movement to a closed configuration) of the jaws (220, 222). Stated differently, jaws (220, 222) would cease to move any further. In another example, controller (312) would adjust one or more of drive inputs (272, 274, 276, 278) in an open direction or closed direction so that the torque being generated returns below the maximum torque threshold without moving the jaws (220, 222) (i.e., returning the torque below the maximum torque threshold in nullspace). Still in yet another example controller (312) would adjust one or more of drive inputs (272, 274, 276, 278) so that the torque being generated returns below the maximum torque threshold by moving the jaws (220, 222) towards the open configuration (i.e., increasing the jaw aperture). Of course, other examples that combine aspects of the aforementioned examples, such as first stopping the directed movement and then moving the jaws (220, 222) towards the open configuration, will be readily apparent to those skilled in the art in view of the teachings herein.

Turning now to FIG. 15 with continuing reference to FIGS. 11, 13B, and 13C, a second example of a method of over-clamping prevention (520) includes calculating (522), by the controller (312), the jaw aperture of the jaws (220, 222). The calculation may be done in real time to provide an ongoing measurement of the jaw aperture. Controller (312) may then receive (524) a request for a new jaw aperture (i.e., commanded aperture), which is a desired distance between the jaws (220, 222).

Afterwards, controller (312) may adjust (526) drive members, namely one or more of drive inputs (272, 274, 276, 278), based on an aperture threshold analysis. In one example, the aperture threshold analysis may include monitoring the ongoing measurement of the jaw aperture as the jaws (220, 222) are moved to the new jaw aperture. During this movement, the difference between the ongoing measurement and the new jaw aperture may be continually calculated. If at any point the difference exceeds a clamping distance threshold, which is a desired difference between the current jaw aperture and a desired jaw aperture, the movement is stopped.

### D. Closed-Loop Clamp Closure System

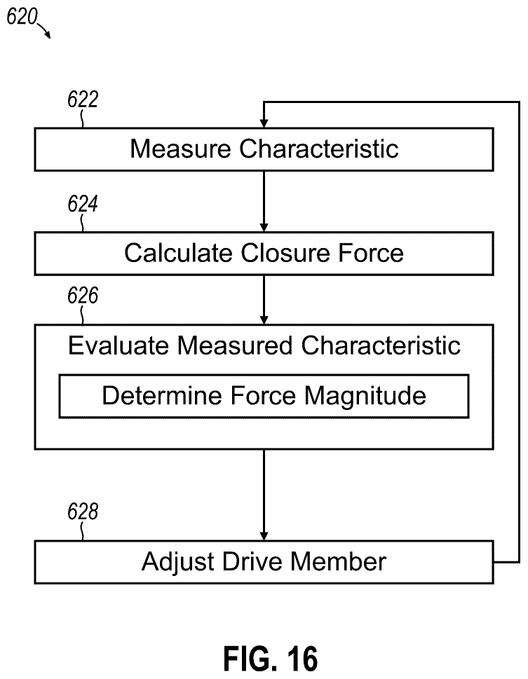

FIG. 16 shows an example of an over-clamping prevention method (620) applicable to clamp closure system (310) suitable for a closed-loop control method. In use, with respect to FIG. 16 and referring back to FIGS. 11, 13B, and 13C, over-clamping prevention method (620) includes measuring, at (622), a desired characteristic of the end effector (218). The characteristic may, in one example, be the torque generated by one or more drive inputs (260, 262, 264, 266) and sensed by one or more torque sensors (272, 274, 276, 278). In other words, one or more characteristic (e.g., torque) measurements may be used in over-clamping prevention method (620).

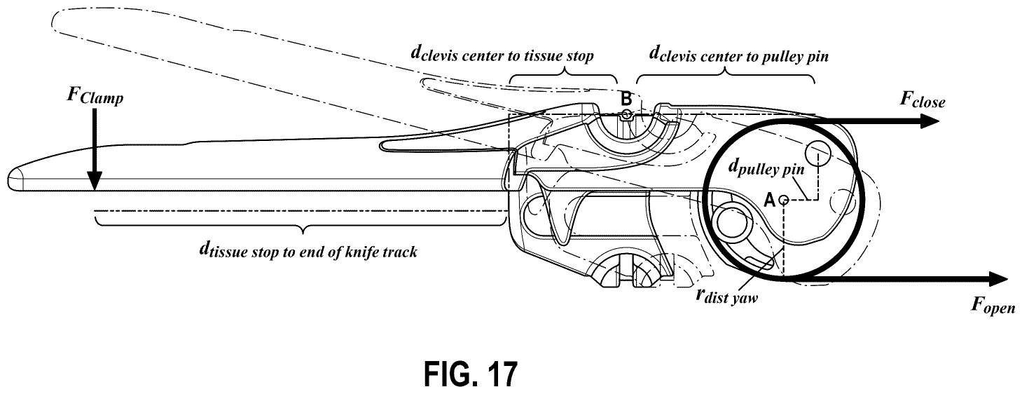

A closure force is then calculated (624) using, for example, the closure force model shown below. To facilitate understanding, corresponding terms of the example of the closure force model are highlighted in FIG. 17.

$${F}_{Clamp Force} = \frac{( {F}_{open} - {F}_{close} )}{2} \times \frac{{r}_{dist yaw}}{{d}_{pulley pin}} \times \frac{{d}_{clevis center to pulley pin}}{{d}_{tissue stop to end of knife track} + {d}_{clevis center to tissue stop}}$$

As can be seen in FIG. 17, forces such as Fₒₚₑₙ and F_{close} are representative of forces being exerted on cables (i.e., cable tension values) connected to the end effector. These forces can be calculated from the torque generated, in whole or in part, by drive inputs (260, 262, 264, 266) and sensed by one or more torque sensors (272, 274, 276, 278).

Then, at (626), the measured characteristic is evaluated. Using torque as an example, an initial force magnitude is determined. In one example, the initial force magnitude is the difference between F_{Clamp Force} and a target closure force, the target closure force being a predetermined setting. The controller (312) then adjusts (628) at least one of the one or more drive inputs (260, 262, 264, 266) in one of the open direction or closed direction. The adjustment aims to decrease the initial force magnitude. In other words, to bring closure force closer to the target closure force. The method (620) loops back to the beginning, where new torque values are measured (622) and received by the controller (312) to calculate (624) a new closure force, F_{Clamp Force, New}. Afterwards, a new force magnitude is calculated using F_{Clamp Force, New} in a similar manner as in (626). Due to the adjustments made to the one or more drive inputs (260, 262, 264, 266), the new force magnitude is smaller than the initial force magnitude. An additional adjustment (628) may be made to at least one of the one or more drive inputs (260, 262, 264, 266) to further decrease the force magnitude. Method (620) may continue repeating, each iteration bringing a calculated closure force of the jaws (220, 222) closer to the target closure force, ensuring an over-clamping clamp force does not occur.

While the above examples refer to preventing an over-clamping clamp force when clamping tissue, such prevention of an over-clamping clamp force applied between jaws (220, 222) similarly applies to closure force, grip force, and generally any instrument configured to engage tissue at jaws or exhibit a closure force. The invention is thus not intended to be unnecessarily limited to clamp force applied to tissue between jaws (220, 222) as shown in the present example.

### IV. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical system, comprising: (a) a surgical instrument, comprising: (i) a shaft assembly; (ii) an end effector including a first jaw and a second jaw movably secured relative to each other such that the first and second jaws are configured to selectively move from an open configuration to a closed configuration; and (iii) a closure assembly operatively connected to the end effector and comprising: (A) a drive member operatively connected to the end effector and configured to selectively move in an open direction or a closed direction to respectively direct one of: (I) the first and second jaws from the open configuration to the closed configuration, and (II) the first and second jaws from the closed configuration to the open configuration, (B) a sensor portion configured to measure a sensed value of a characteristic of the drive member, and (C) a controller configured to: (I) receive the sensed value, (II) evaluate the received sensed value, and (III) adjust the movement of the drive member based on the evaluation.

### Example 2

The surgical system of Example 1, the drive member operatively connected to the end effector with a cable, wherein the evaluation comprises comparing the received sensed value against a threshold value, the threshold value being a value of the characteristic that corresponds to a predetermined tension of the cable.

### Example 3

The surgical system of any one or more of Examples 1 through 2, wherein the characteristic is a torque being generated by the drive member.

### Example 4

The surgical system of any one or more of Examples 1 through 2, wherein the characteristic is a current being drawn by the drive member.

### Example 5

The surgical system of any one or more of Examples 1 through 4, the shaft assembly comprising an articulation section configured to articulate to a selected articulation, wherein the end effector is operatively connected to the articulation section and distally extending from the articulation section, and wherein the drive member is further configured to direct the articulation section to the selected articulation.

### Example 6

The surgical system of Example 5, wherein threshold value is based on the selected articulation.

### Example 7

The surgical system of any one or more of Examples 2 through 6, the comparison of the received sensed value against the threshold value further comprising determining whether the received sensed value meets or exceeds the threshold value for a period of time.

### Example 8

The surgical system of any one or more of Examples 1 through 7, wherein adjusting the movement of the drive member comprises stopping the movement.

### Example 9

The surgical system of any one or more of Examples 1 through 8, wherein adjusting the movement of the drive member comprises receiving an additional sensed value, the additional sensed value not exceeding the threshold value.

### Example 10

The surgical system of any one or more of Examples 1 through 9, wherein adjusting the movement of the drive member does not move the end effector.

### Example 11

The surgical system of any one or more of Examples 1 through 8, wherein adjusting the movement of the drive member further comprises, before receiving the additional sensed value, moving the drive member in the open direction.

### Example 12

The surgical system of Example 1, the controller further configured to calculate a closure force based on the sensed value, wherein the evaluation comprises determining a first force magnitude based on the difference between the closure force and a target closure force, and wherein adjusting the movement of the drive member comprises: (a) receiving an additional sensed value; (b) calculating an additional closure force based on the additional sensed value; and (c) determining a second force magnitude based on the difference between the additional closure force and the target closure force, the second force magnitude being less than the first force magnitude; wherein before receiving the additional sensed value, the drive member is moved in one of the open direction and the closed direction.

### Example 13

The surgical system of Example 1, the drive member operatively connected to the end effector with a cable, wherein the evaluation comprises: (a) comparing the received sensed value against a threshold value, the threshold value being a value of a torque being generated by the drive member that corresponds to a predetermined tension of the cable; and (b) determining whether the received sensed value meets or exceeds the threshold value for a period of time.

### Example 14

The surgical system of Example 13, wherein adjusting the movement of the drive member comprises stopping the movement.

### Example 15

The surgical system of Example 13, wherein adjusting the movement of the drive member does not move the end effector and comprises receiving an additional sensed value, the additional sensed value not exceeding the threshold value.

### Example 16

A surgical system, comprising a surgical instrument, comprising: (a) an end effector including a first jaw and a second jaw movably secured relative to each other such that the first and second jaws are configured to selectively move from an open configuration to a closed configuration, a distance between the first and second jaws defining an aperture; and (b) a closure assembly operatively connected to the end effector and comprising: (i) a drive member operatively connected to the end effector and configured to selectively move in an open direction or a closed direction to respectively direct the first and second jaws from the open configuration to the closed configuration, and (ii) a controller configured to: (A) calculate the aperture, (B) receive a commanded aperture defining a desired distance between the first and second jaws, (C) adjust the movement of the drive member based on the commanded aperture, and (D) stop the movement of the drive member if the magnitude of the difference between the aperture and the commanded aperture is greater than a clamping distance threshold.

### Example 17

A method for reducing a closure force imposed on a tissue by a surgical instrument, comprising: (a) measuring a sensed value of a characteristic of a drive member operatively connected to an end effector of the surgical instrument, the drive member configured to selectively move in an open direction or a closed direction to respectively direct one of: (i) a first jaw and a second jaw of the end effector from an open configuration to a closed configuration, the first and second jaws movably secured relative to each other such that the first and second jaws are configured to selectively move from the open configuration to the closed configuration, and (ii) the first and second jaws from the closed configuration to the open configuration, (b) evaluating the sensed value; and (c) adjusting the movement of the drive member based on the evaluation.

### Example 18

The method of Example 17, wherein evaluating the sensed value comprises comparing the sensed value against a threshold value, the threshold value being a value of the characteristic that corresponds to a predetermined tension of a cable operatively connecting the drive member to the end effector.

### Example 19

The method of Example 18, wherein comparing the sensed value against the threshold value further comprising determining whether the sensed value meets or exceeds the threshold value for a period of time.

### Example 20

The method of Example 17, further comprising calculating a closure force based on the sensed value, wherein evaluating the sensed value comprises determining a first force magnitude based on the difference between the closure force and a target closure force, and wherein adjusting the movement of the drive member comprises: (a) receiving an additional sensed value; (b) calculating an additional closure force based on the additional sensed value; and (c) determining a second force magnitude based on the difference between the additional closure force and the target closure force, the second force magnitude being less than the first force magnitude; wherein before receiving the additional sensed value, the drive member is moved in one of the open direction and the closed direction.

### Example 21

The method of Example 19, wherein adjusting the movement of the drive member comprises receiving an additional sensed value, the additional sensed value not exceeding the threshold value.

### Example 22

The method of Example 21, wherein adjusting the movement of the drive member does not move the end effector.

### Example 23

The method of Example 21, wherein adjusting the movement of the drive member comprises, before receiving the additional sensed value, moving the drive member in the open direction.

### V. Miscellaneous

For clarity of disclosure, the terms “proximal” and “distal” are defined herein relative to a surgeon or other operator grasping a surgical instrument having a distal surgical end effector. The term “proximal” refers to the position of an element closer to the surgeon or other operator and the term “distal” refers to the position of an element closer to the surgical end effector of the surgical instrument and further away from the surgeon or other operator. Moreover, terms such as “upper” and “lower” are merely spatial terms relative to the figures and are not intended to unnecessarily limit the invention described herein.

It should be noted that the terms “couple,” “coupling,” “coupled” or other variations of the word couple as used herein, along with the terms “connected” or “connecting” may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of instruments having RF electrodes, it should be understood that various teachings herein may be readily applied to a variety of other types of devices. By way of example only, the various teachings herein may be readily applied to other types of surgical instruments including tissue graspers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical system, comprising:
(a) a surgical instrument, comprising:
(i) a shaft assembly;
(ii) an end effector including a first jaw and a second jaw movably secured relative to each other such that the first and second jaws are configured to selectively move from an open configuration to a closed configuration; and
(iii) a closure assembly operatively connected to the end effector and comprising:
(A) a drive member operatively connected to the end effector and configured to selectively move in an open direction or a closed direction to respectively direct one of:
(I) the first and second jaws from the open configuration to the closed configuration, and
(II) the first and second jaws from the closed configuration to the open configuration,
(B) a sensor portion configured to measure a sensed value of a characteristic of the drive member, and
(C) a controller configured to:
(I) receive the sensed value,
(II) evaluate the received sensed value, and
(III) adjust the movement of the drive member based on the evaluation.

2. The surgical system of claim 1, the drive member operatively connected to the end effector with a cable, wherein the evaluation comprises comparing the received sensed value against a threshold value, the threshold value being a value of the characteristic that corresponds to a predetermined tension of the cable.

3. The surgical system of claim 2, wherein the characteristic is a torque being generated by the drive member.

4. The surgical system of claim 2, wherein the characteristic is a current being drawn by the drive member.

5. The surgical system of claim 2, the shaft assembly comprising an articulation section configured to articulate to a selected articulation, wherein the end effector is operatively connected to the articulation section and distally extending from the articulation section, and wherein the drive member is further configured to direct the articulation section to the selected articulation.

6. The surgical system of claim 5, wherein threshold value is based on the selected articulation.

7. The surgical system of claim 2, the comparison of the received sensed value against the threshold value further comprising determining whether the received sensed value meets or exceeds the threshold value for a period of time.

8. The surgical system of claim 7, wherein adjusting the movement of the drive member comprises stopping the movement.

9. The surgical system of claim 7, wherein adjusting the movement of the drive member comprises receiving an additional sensed value, the additional sensed value not exceeding the threshold value.

10. The surgical system of claim 9, wherein adjusting the movement of the drive member does not move the end effector.

11. The surgical system of claim 9, wherein adjusting the movement of the drive member further comprises, before receiving the additional sensed value, moving the drive member in the open direction.

12. The surgical system of claim 1, the controller further configured to calculate a closure force based on the sensed value, wherein the evaluation comprises determining a first force magnitude based on the difference between the closure force and a target closure force, and wherein adjusting the movement of the drive member comprises:
(a) receiving an additional sensed value;
(b) calculating an additional closure force based on the additional sensed value; and
(c) determining a second force magnitude based on the difference between the additional closure force and the target closure force, the second force magnitude being less than the first force magnitude;
wherein before receiving the additional sensed value, the drive member is moved in one of the open direction and the closed direction.

13. The surgical system of claim 1, the drive member operatively connected to the end effector with a cable, wherein the evaluation comprises:
(a) comparing the received sensed value against a threshold value, the threshold value being a value of a torque being generated by the drive member that corresponds to a predetermined tension of the cable; and
(b) determining whether the received sensed value meets or exceeds the threshold value for a period of time.

14. The surgical system of claim 13, wherein adjusting the movement of the drive member comprises stopping the movement.

15. The surgical system of claim 13, wherein adjusting the movement of the drive member does not move the end effector and comprises receiving an additional sensed value, the additional sensed value not exceeding the threshold value.

16. A surgical system, comprising a surgical instrument, comprising:
(a) an end effector including a first jaw and a second jaw movably secured relative to each other such that the first and second jaws are configured to selectively move from an open configuration to a closed configuration, a distance between the first and second jaws defining an aperture; and
(b) a closure assembly operatively connected to the end effector and comprising:
(i) a drive member operatively connected to the end effector and configured to selectively move in an open direction or a closed direction to respectively direct the first and second jaws from the open configuration to the closed configuration, and
(ii) a controller configured to:
(A) calculate the aperture,
(B) receive a commanded aperture defining a desired distance between the first and second jaws,
(C) adjust the movement of the drive member based on the commanded aperture, and
(D) stop the movement of the drive member if the magnitude of the difference between the aperture and the commanded aperture is greater than a clamping distance threshold.

17. A method for reducing a closure force imposed on a tissue by a surgical instrument, comprising:
(a) measuring a sensed value of a characteristic of a drive member operatively connected to an end effector of the surgical instrument, the drive member configured to selectively move in an open direction or a closed direction to respectively direct one of:
(i) a first jaw and a second jaw of the end effector from an open configuration to a closed configuration, the first and second jaws movably secured relative to each other such that the first and second jaws are configured to selectively move from the open configuration to the closed configuration, and
(ii) the first and second jaws from the closed configuration to the open configuration,
(b) evaluating the sensed value; and
(c) adjusting the movement of the drive member based on the evaluation.

18. The method of claim 17, wherein evaluating the sensed value comprises comparing the sensed value against a threshold value, the threshold value being a value of the characteristic that corresponds to a predetermined tension of a cable operatively connecting the drive member to the end effector.

19. The method of claim 18, wherein comparing the sensed value against the threshold value further comprising determining whether the sensed value meets or exceeds the threshold value for a period of time.

20. The method of claim 17, further comprising calculating a closure force based on the sensed value, wherein evaluating the sensed value comprises determining a first force magnitude based on the difference between the closure force and a target closure force, and wherein adjusting the movement of the drive member comprises:
(a) receiving an additional sensed value;
(b) calculating an additional closure force based on the additional sensed value; and
(c) determining a second force magnitude based on the difference between the additional closure force and the target closure force, the second force magnitude being less than the first force magnitude;
wherein before receiving the additional sensed value, the drive member is moved in one of the open direction and the closed direction.
